(19) United States
(12) Patent Application Publication
(10) Pub. No.: US 2011/0165267 A1
(43) Pub. Date: Jul. 7, 2011

(54) AGENTS FOR TREATING AND/OR AVOIDING FIRE BLIGHT

(75) Inventors: Frank Saalfeld, Kurten-Durscheid (DE); Ulf Abele, Hohenwart (DE); Hans Siebenlist, Schweitenkirchen (DE)

(73) Assignee: LANXESS DISTRIBUTION GMBH, Langenfeld (DE)

(21) Appl. No.: 12/867,523

(22) PCT Filed: Feb. 12, 2009
(86) PCT No.: PCT/EP09/51606

§ 371 Date: Mar. 22, 2011

(30) Foreign Application Priority Data
Feb. 15, 2008 (DE) 08151535.5
Mar. 26, 2008 (DE) 08153287.1

(51) Int. Cl. A01N 59/08; A01P 1/00; C07C 59/265; C07C 53/06

(52) U.S. Cl. 424/678; 562/584; 562/512

(57) ABSTRACT

The invention relates to agents for treating and/or avoiding fire blight in plants of the Rosaceae, in particular of the subfamilies Maloideae and Rosoideae, containing formiates as the active component.

The invention relates to compositions for the treatment and/or prevention of fire blight in plants of the family Rosaceae, in particular the subfamilies Maloideae and Rosoideae, comprising formates as the active constituent.

Fire blight is a plant disease caused by the bacterium Erwinia amylovora which causes great commercial damage in particular in fruit production because it spreads rapidly; to date, it has been very difficult to control.

The range of host plants is limited to plants from the family Rosaceae (rose family). The pathogen has been detected in a large number of subfamilies, genera and species of this family, in particular in the subfamilies Maloideae and Rosoideae. As regards northern and central Europe, the cultured and ornamental forms of apple (Malus) and pear (Pyrus) and also pyracantha (Pyracantha), quince (Cydonia), stranvesia (Stranvesia), Sorbus species such as sorb apple (Sorbus), white- and red-flowered hawthorn (Crategus), Japanese quince (Chaenomeles), cotoneaster (Cotoneaster), loquat (Eriobotrya) and medlar (Mespilus) are frequently affected. The severity of the disease depends greatly on the weather conditions during the flowering phase of the variety in question.

The bacteria spend the dormant period of the vegetation in diseased bark sections, from where they are dispersed in spring and summer via raindrops, wind and sucking and flower-visiting insects such as honeybees, aphids, psyllids, wasps, bumblebees and flies. Usually, the disease starts with a floral infection, also referred to hereinbelow as primary fire blight, which, as the year goes by, can result in a massive attack on other plant parts, such as, for example, shoots. Important entrance portals for the pathogens are open blossom and wounds, and in the case of sufficient moisture also natural openings such as nectar glands, stomata, lenticelles and scars of leaf petioles and of peduncles. This infection of other plant parts, which happens at a later point in time, is also referred to hereinbelow as secondary fire blight.

Typical features of the fire blight disease are in most cases dark-brown to black stems of the dead blossom and leaves on the diseased branches, and the main vascular bundles of the leaves are frequently noticeably dark in color. The dead, desiccated leaves and fruits remain hanging on the trees. The diseased young shoot tips, which are initially pale green in appearance, frequently bend in a crook-like fashion as the result of lack of water. In the case of late-flowering woody species and second flushes, the risk of infection is high even in summer. Shoots can also be infected over the entire vegetation period, in particular before the end of shooting. In spring and summer, moist, initially colorless, but later brown, sticky droplets are observed on infected shoots, fruits and rootstock. This infectious bacterial mucus (exudate) is produced in plentiful amounts, in particular under humid-warm conditions. Besides the droplets, filiform structures may also occur. Small or larger dead patches on the trunks and stronger branches can be observed in winter, but frequently also as early as summer or autumn. (“Der Feuerbrand gefährdet Obst- and Ziergehölze” [Fire blight endangers woody fruit and ornamental species], Pflanzenschutzdienst [Plant protection service], Landesanstalt für Pflanzenschutz [Regional institute for plant protection], Stuttgart 2000/2003).

In practice, the application of conventional plant protection products is rather unsuccessful. Thus, for example, resistance inductors, bacterial antagonists, rocks, disinfectants, fungicides with a bacterial side effect and combinations of such products have been tested. None of these compositions has shown a sufficient effect in this context. (E. Moltmann,

“Feuerbrandbekämpfung in Baden-Württemberg” [The control of fire blight in Baden-Württemberg], Festschrift 50 Jahre Landesanstalt für Pflanzenschutz Stuttgart 2005).

A wide range of products for controlling fire blight is furthermore known from the literature:

EP-A 0 158 074 discloses the use, for the control of fire blight, of benzisothiazo dioxides which are known as sweeteners.

WO 02/052942 describes the control of fire blight by slaked lime, preferably in the form of an aqueous solution.

CA-A-2291984 and U.S. Pat. No. 4,569,841 disclose the control of fire blight by Erwinia herbicola bacteria, WO 2005/048717 the application of other bacteria or fungi for this purpose.

EP-A 0 565 266 discloses a combination of a copper complex and a partially neutralized water-soluble polycarboxylic acid for controlling plant diseases caused by bacteria or fungi.

EP-A 1 075 185 proposes a combination of dodine and anilinopyridine for controlling fire blight.

DD-A 273 192 discloses the use of the strepithricin antibiotic nourseothricin for controlling fire blight, and GB-A 1315430 the use of an antibiotic “A-201A”.

DE-A 3640048 describes nitroalkanols for controlling fire blight and also mentions that the use of disinfectants such as benzalkonium chloride is suitable for destroying blossom and therefore eliminates the most important infection opportunity for fire blight.

EP-A 1300078 proposes natural herbal components selected from tea tree oil, oil of wintergreen and eugenol or a mixture of these for controlling fire blight.

U.S. Pat. No. 5,686,389 proposes the use of 5-hydroxylysine or 1,4-diaminobutanone for controlling fire blight and maintains that these active substances are also effective against antibiotic-resistant strains.

JP-A 1090102 discloses β-thujaplicin as an agent against fire blight.

JP-A 63099005 proposes poloixin antibiotics in combination with an imidazole compound for this purpose, while JP-A-57081404 proposes alkoxycarbonyl ureidophenyl.

GP-A-1049116 discloses a tetrahydroxypyrimidine derivative in aqueous solution as composition against fire blight which is allegedly not phytotoxic.

GB 1421 615 describes the use of propionic acid against Erwinia amylovora.

However, these products, too, do not show satisfactory results in the control of fire blight.

The only active substance which has been employed widely and successfully to date is the antibiotic streptomycin, which is normally used in the form of its sulfate. The disadvantage here that, due to residue formation, its use is subject to severe legal restrictions in some countries and banned completely in others.

There is therefore an urgent need for a composition for controlling fire blight which firstly has a high efficacy and secondly does not have the abovementioned disadvantages. Against this background, the object of the present invention is to provide a composition for preventing and/or treating fire blight.

Thus, the invention relates to aqueous plant treatment compositions comprising
at least 85% by weight, preferably at least 90% by weight, especially preferably at least 94% by weight, of water
from 0.1% by weight to 3% by weight, preferably 0.2% by weight to 2% by weight, of calcium salts based on calcium oxide
from 0.15% by weight to 4.5% by weight, preferably from 0.3% by weight to 3% by weight, of formates, based on formic acid,
   where
the pH of the aqueous plant treatment compositions is 3.0 to 6.5, based on standard conditions.

It should be noted here that the scope of the invention comprises any possible components, value ranges or methodological parameters mentioned hereinabove and hereinbelow, in general terms or in preferred ranges.

In a preferred embodiment, the molar ratio of formates to calcium is between 1:4 and 4:1, preferably between 1:2 and 4:1, especially preferably between 1:1.5 and 3:1 and very especially preferably between 1:1 and 2.5:1.

In a further preferred embodiment, the pH of the compositions according to the invention is from 4.0 to 5.0 and especially preferably from 4.0 to 4.8 and very especially preferably from 4.0 to 4.3 under standard conditions.

Acids and acidic salts are suitable for obtaining the abovementioned pH values. Especially suitable are organic acids which have a pKa of from 2 to 6 and which do not form sparingly soluble salts with calcium ions, and suitably acidic ammonium salts such as, for example, ammonium chloride and organic ammonium salts or adducts such as, for example, alkali metal diformates. Sparingly soluble calcium salts are understood as meaning, for the purposes of the invention, those which have a solubility of less than 1 g/l in the presence of the corresponding free acid and at pH values of from 3.0 to 6.5. Examples of suitable organic acids are formic acid, acetic acid, propionic acid, lactic acid, tartaric acid, malic acid, succinic acid and citric acid.

To treat the plants, the plant treatment compositions according to the invention are preferably applied to the plant organs in the customary manner, for example by spraying or brushing on, preferably by spraying. The application can be carried out preventatively or even on plants which have already been infected with fire blight.

By way of example and with preference, the plant treatment compositions according to the invention are applied at application rates of from 50 to 1000 l/ha, preferably from 300 to 600 l/ha, especially preferably approximately 500 l/ha per application.

Furthermore, it is preferred to apply between 1 kg and 20 kg of calcium salts, based on calcium oxide, and between 1.5 kg and 30 kg of formates, based on formic acid, per application and hectare.

To control primary fire blight, it is possible, for example in the case of stone fruit, to carry out 1 to 5, preferably 2 to 3, applications during flowering.

To control secondary fire blight, it is possible to carry out, for example in the case of stone fruit, 1 to 10, preferably 3 to 8 and especially preferably 5 to 6 applications from the red-bud stage up to the pre-harvest stage.

Typically, higher calcium and formate concentrations will be chosen for controlling primary fire blight than for controlling secondary fire blight. Thus, it is preferred to apply between 5 kg and 20 kg of calcium salts, based on calcium oxide, and between 7.5 kg and 30 kg of formates, based on formic acid, per application and hectare for controlling primary fire blight, while it is preferred to apply between 1 kg and 6 kg of calcium salts, based on calcium oxide, and between 1.5 kg and 10 kg of formates, based on formic acid, per application and hectare for controlling secondary fire blight.

To make the application as simple as possible for the user, the aqueous plant treatment compositions according to the invention are preferably prepared such that essentially solid formulations, or at least essentially liquid concentrates, are diluted with the appropriate amount of water.

The at least essentially solid formulations, which are also encompassed within the scope of the invention, typically comprise
between 5 and 40% by weight of calcium salts, based on calcium oxide, preferably between 10 and 35% by weight and especially preferably between 20 and 30% by weight
between 5 and 70% by weight of inorganic formates, based on formic acid, preferably between 20 and 60% by weight and especially preferably between 45 and 60% by weight
between 0.5 and 70% by weight of an organic acid which is essentially solid under standard conditions or an acidic salt with a pKa of from 2 to 6.

In a preferred embodiment, the molar ratio of formate to calcium is between 1:4 and 4:1, preferably between 1:2 and 4:1, especially preferably between 1:1.5 and 3:1, and very especially preferably between 1:1 and 2.5:1.

The calcium and the formates can, in principle, be introduced into the aqueous plant treatment compositions according to the invention and into the essentially solid formulations and essentially liquid concentrates in the form of any compound, as long as they do not have any significant phytotoxic effect, apart from the desired control of Erwinia amylovora. This, however, can be tested in simple tests with which a person skilled in the art is sufficiently familiar. Preferred substances for the introduction of formates are alkali metal formates such as, for example, potassium formate and sodium formate, alkali metal diformates such as, for example, potassium diformate and sodium diformate, and alkaline-earth metal formates such as, for example, calcium formate, or mixtures of such formates. Substances which are preferred for the introduction of calcium are calcium formate and other calcium salts of organic carboxylic acids, calcium chloride, calcium nitrate and other inorganic calcium salts, with calcium formate and calcium chloride being preferred and calcium formate being especially preferred.

In an especially preferred embodiment, the at least essentially solid formulations comprise from 10 to 98, preferably 30 to 90, and especially preferably 50 to 80% by weight of calcium formate. In a further preferred embodiment, the at least essentially solid formulations additionally comprise between 0.5 and 70% by weight, preferably 1 to 30 and especially preferably 5 to 20% by weight of an organic acid which is essentially solid under standard conditions and has a pKa of from 2 to 6, preferably 3 to 5, especially preferably 3 to 4, such as, for example citric acid.

The at least essentially solid formulations can be formulated and packaged for example as powders, pellets, granules, capsules or tablets, if desired also in portions; they are preferably formulated as granules, even more preferably as essentially dust-free microgranules. The at least essentially solid formulations can then be diluted to the use concentration with water immediately prior to use, or first diluted with water, giving rise to at least essentially liquid concentrates which, in turn, can then be diluted to the use concentration with water. The abovementioned parameters and concentrations for aqueous plant treatment compositions then apply analogously to the at least essentially solid formulations which are diluted with water. The ranges and preferred ranges which have been mentioned for the at least essentially solid formulations apply analogously to the at least essentially liquid concentrates. Preferred, at least essentially liquid concentrates are those which can be obtained by mixing in each case one part by weight of the essentially solid formulations according to the invention and 0.2 to 20, preferably 0.5 to 10, especially preferably 0.5 to 5 and very especially preferably 1 to 5 parts by weight of water. The abovementioned at least essentially liquid concentrates are also encompassed by the invention.

Formulations which are excluded from the invention are those which comprise from 70 to 80% by weight of calcium formate, 14 to 18% by weight of calcium chloride and 1 to 15% by weight of citric acid, and those aqueous plant treatment compositions and concentrates which are obtainable therefrom by dilution with water.

Furthermore excluded from the invention are concentrates which comprise calcium formate, where the total calcium content is 260 g/l based on calcium oxide and where the concentrates furthermore comprise 1.5% by weight of manganese in the form of manganese salts and 0.5% zinc in the form of zinc salts and the concentrates have a pH of 5.5. Likewise excluded from the invention are aqueous plant treatment compositions which can be obtained by diluting the abovementioned concentrates with water.

The aqueous plant treatment compositions according to the invention, the at least essentially solid formulations and the at least essentially liquid concentrates may furthermore comprise various additives. As regards the additives mentioned hereinbelow, in each case independently of one another, it is also possible that they are not present. The statements made hereinbelow for the at least essentially solid formulations also apply analogously to the aqueous plant treatment compositions according to the invention and the essentially liquid concentrates, taking account of suitable dilution.
Hygroscopic substances and/or humectants for regulating the moisture content: hygroscopic substances are, for example, hygroscopic inorganic salts such as, for example, calcium chloride or calcium nitrate; suitable humectants are, for example, organic substances such as, for example, glycerol, polydextrose, sorbitol, xylitol, propylene glycols, polyethylene glycols or mixtures of these polyols.
In this context, the at least essentially solid formulations may comprise for example from 0.01 to 40, preferably from 0.2 to 20 and especially preferably from 0.5 to 2% by weight of hygroscopic substances and/or humectants.
Surface-active substances such as, for example, surfactants. Surfactants may, for example, be nonionic, cationic and amphoteric surfactants, preferably anionic surfactants. Suitable anionic surfactants are, for example, alkyl sulfates, alkyl ether sulfates, alkylarylsulfonates, alkyl succinates, alkyl sulfosuccinates, N-alkyl sarcosinates, acyl taurates, acyl isethionates, alkyl phosphates, alkyl ether phosphates, alkyl ether carboxylates, alpha-olefinsulfonates, in particular the alkali and alkaline-earth metal salts, for example sodium, potassium, magnesium, calcium, and ammonium salts and triethanolamine salts. The alkyl ether sulfates, alkyl ether phosphates and alkyl ether carboxylates may in each case have for example between 1 and 10 ethylene oxide or propylene oxide units, preferably 1 to 3 ethylene oxide units. Suitable examples are sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate. In this context, the at least essentially solid formulations may comprise for example from 0.01 to 10, preferably from 0.2 to 8, especially preferably from 0.3 to 5 and very especially preferably from 0.5 to 3% by weight of surface-active substances.
Wetting agents, such as, for example, alkali metal salts, alkaline-earth metal salts, ammonium salts of aromatic sulfonic acids, for example ligno-, phenol-, naphthalene- and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols or fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenol or tributylphenyl polyglycol ethers, alkylaryl polyether alcohols, isotridecyl alcohol, fatty alcohol/ethylene oxide condensates, ethoxylated castor oil, polyoxyethylene alkyl ethers or polyoxypropylene, lauryl alcohol polyglycol ether acetate, sorbitol esters, lignosulfite waste liquors or methylcellulose. In this context, the at least essentially solid formulations may comprise, for example, from 0.01 to 8, preferably 0.2 to 6, especially preferably 0.3 to 5 and very especially preferably 0.5 to 3% by weight of wetting agents.
Emulsifiers such as, for example, sodium salts, potassium salts and ammonium salts of straight-chain aliphatic carboxylic acids of chain length C₁₂-C₂₀, sodium hydroxyocta-decanesulfonate, sodium salts, potassium salts and ammonium salts of hydroxy fatty acids of chain length C₁₂-C₂₀ and their sulfation or acetylation products, alkyl sulfates, also as triethanolamine salts, (C₁₀-C₂₀)-alkylsulfonates, (C₁₀-C₂₀)-alkylarylsulfonates, dimethyl-di(C₈-C₁₈)-alkylammonium chloride, acyl-, alkyl-, oleyl- and alkylaryloxethylates and their sulfation products, alkali metal salts of the sulfosuccinic esters with aliphatic saturated monohydric alcohols of chain length C₄-C₁₆, sulfosuccinic 4-esters with polyethylene glycol ethers of monohydric aliphatic alcohols of chain length C₁₀-C₁₂ (disodium salt), sulfosuccinic 4-esters with polyethylene glycol nonylphenyl ether (disodium salt), biscyclohexyl sulfosuccinate (sodium salt), lignosulfonic acid and its calcium, magnesium, sodium and ammonium salts, polyoxyethylene sorbitan monooleate with 20 ethylene oxide groups, resin acids, hydrogenated and dehydrogenated resin acids and their alkali metal salts, dodecylated sodium diphenyl ether disulfonate, and copolymers of ethylene oxide and propylene oxide with a minimum content of 10% by weight of ethylene oxide. The following are preferably used as emulsifiers: sodium lauryl sulfate, sodium lauryl ether sulfate, ethoxylated (3 ethylene oxide groups); the polyethylene glycol(4-20) ethers of oleyl alcohol and the polyethylene oxide(4-14) ethers of nonylphenol. In this context, the at least essentially solid formulations may comprise for example from 0.01 to 15, preferably 0.2 to 8, especially preferably 0.5 to 6 and very especially preferably 1 to 5% by weight of emulsifiers.
Dispersants such as, for example, alkylphenyl polyglycol ethers. In this context, the at least essentially solid formulations may comprise for example from 0.01 to 8, preferably 0.1 to 6, especially preferably 0.2 to 5 and very especially preferably 0.4 to 3% by weight of dispersants.
Stabilizers such as, for example, cellulose and cellulose derivatives. In this context, the at least essentially solid formulations may comprise for example from 0.01 to 6, preferably 0.01 to 3, especially preferably 0.01 to 2 and very especially preferably 0.01 to 1% by weight of stabilizers.
Stickers, such as carboxymethylcellulose, natural and synthetic pulverulent, granular or latexlike polymers, such as gum arabic, polyvinyl alcohol, polyvinyl acetate, and natural phospholipids, such as cephalins and lecithins, and synthetic phospholipid, and also liquid paraffins. In this context, the at least essentially solid formulations may comprise for example from 0.01 to 8, preferably 0.1 to 4, especially preferably 0.2 to 3 and very especially preferably 0.2 to 2% by weight of stickers.
Spreaders such as, for example, isopropyl myristate, polyoxyethylene nonylphenyl ether and polyoxyethylene laurylphenyl ether. In this context, the at least essentially solid formulations may comprise for example from 0.01 to 20, preferably 0.1 to 10, especially preferably 0.1 to 5 and very especially preferably 0.1 to 2% by weight of spreaders.
Organic solvents such as, for example, mono- or polyhydric alcohols, esters, ketones and hydrocarbons. Examples of suitable solvents are paraffins, for example mineral oil fractions, mineral and vegetable oils, butanol or glycol and their ethers and esters, ketones such as acetone, methyl ethyl ketone, methyl isobutyl ketone or cyclohexanone. In this context, the at least essentially solid formulations may comprise for example from 0.01 to 25, preferably 0.2 to 12, especially preferably 0.5 to 7 and very especially preferably 1 to 4% by weight of organic solvents.
Fragrances and dyes such as inorganic pigments, for example iron oxide, titanium oxide,

Prussian Blue and organic dyes such as alizarin, azo and metal phthalocyanine dyes and trace nutrients such as salts of iron, manganese, boron, copper, cobalt, molybdenum and zinc. In this context, the at least essentially solid formulations may comprise for example in each case from 0.001 to 4, preferably 0.01 to 1, especially preferably 0.01 to 0.8% by weight of fragrances and dyes.
Anti-dust agents, such as, for example, polyglycols and polyglycol ethers. In this context, the at least essentially solid formulations may comprise in each case from 0.01 to 2, preferably 0.05 to 1, especially preferably 0.1 to 0.5% by weight of anti-dust agents.
Buffer substances, buffer systems or pH regulators. In this context, the at least essentially solid formulations may comprise for example in each case from 0.01 to 10, preferably 0.1 to 5% by weight of buffer substances, buffer systems or pH regulators.

Preferably, the content of the abovementioned additives in the solid formulations is in total 0.01 to 25, preferably 0.5 to 10 and especially preferably 1 to 5% by weight.

Furthermore, the at least essentially solid formulations may comprise further active components such as bactericides, fungicides, insecticides, acaricides and growth regulators. Preferably, the at least essentially solid formulations contain no, or from 0.001 to 10% by weight of, further active components, preferably no, or from 0.001 to 2% by weight of, further active components, but they especially preferably do not contain any further active components.

Especially preferred essentially solid formulations are those which comprise between 50 and 80, preferably 60 and 75% of calcium formate and over 15%, preferably over 16% by weight and especially 18 to 25% by weight of a polybasic organic acid such as, in particular, citric acid, and, if appropriate, but preferably, a wetting agent or a humectant.

The aqueous plant treatment compositions according to the invention, the at least essentially solid formulations and the at least essentially liquid concentrates are suitable in particular for use in a method for controlling and/or preventing fire blight in all plants which are susceptible to fire blight such as, in particular, plants of the family Rosaceae, and here in particular woody species, and here preferably fruit trees such as apple, pear and quince, but also white- and red-flowering hawthorn, cotoneaster and medlars.

The examples which follow illustrate the invention is greater detail.

### EXAMPLES

1. Testing the Reduction of the Erwinia amylovora Population on Apple Leaves

Test plants: Apple plants cv. Jonagold on M9 rootstock.

The three youngest fully-unfolded leaves on growing shoots were inoculated with the fire blight pathogen. Three shoots were used per treatment. During the experiment, the plants were incubated at 27°/20° (day/night) and 12 h light at 70-90% relative atmospheric humidity.

Pathogen: Erwinia amylovora 385 was grown on an agar plate. The apple leaves were inoculated with 10⁶ bacteria/leaf.

Plant Treatment Composition:

1 part by weight of a solid formulation comprising

75% by weight of calcium formate

10% by weight of citric acid

13% by weight of calcium chloride

2% by weight of further adjuvants, including a wetter, was employed in in each case 100 parts by weight (P 1%), 50 parts by weight (P 2%) and 16.7 parts by weight (P 6%) of water and the solution was employed for the subsequent treatment.

Treatments:

The treatments were carried out two hours after the inoculation, to runoff-point on the dried leaf.

(1) P 1%

(2) P 2%

(3) Water as the control

(4) P 6%

Sampling

Sampling was carried out 90 minutes (before the treatment) and 24 hours after applying the pathogen. A sample consisted of in each case two leaves of a treatment. Three samples were taken from each treatment. The test was repeated once. The apple leaves were washed, and a serial dilution series of the wash water was plated onto agar medium.

Evaluation

After the agar plate had incubated for 48 h at 27° C. in the dark, the colony-forming units (cfu) were determined. The lower detection limit for determining the live cell count with this method was 100 bacteria/leaf. By comparing the treatments with the control, which had been sprayed with water, it was possible to calculate the efficacy. The data of the six results of each treatment were averaged.

1×10⁶ cells of E. amylovora were applied to each leaf. 1.5 h thereafter, it was possible to reisolate on average 8×10⁵ cells. Thereafter, the preparations were sprayed on. 24 h after the inoculation, an average of 8740 cells could still be detected on the leaves which had been treated with water. On the leaves which had been treated with P 6%, fire blight pathogens were no longer detectable after 24 h (Table 1). After the treatment with P 2%, live fire blight bacteria were detected in two of the 6 samples. In total, however, the number of pathogens was reduced by 99%. Treatment with P 1% reduced the epiphytic fire blight pathogens by 96%. Here, the pathogen could be detected in one sample.

TABLE 1
Activity of the plant treatment composition against the
fire blight pathogen on apple leaves cv. Jonagold.

| Time after | Untreated | P 6% | P 2% | P 1% |
|---|---|---|---|---|
| inoculation | cfu/leaf | cfu/leaf | cfu/leaf | cfu/leaf |
| Mean after 1.5 h | 80 000 | — | — | — |
| Mean after 24 h | 8740 | 0 | 86 | 356 |
| Activity (%) | | 100% | 99% | 96% |

2. Testing the Reduction of the Erwinia amylovora Population on Apple Blossom

Test plants: Apple blossom cv. Gala

Apple blossom cv. Gala were used in the following tests. The trees had not been treated with plant protection compositions. The flowers were placed by the petiole into a 10% strength sucrose solution and incubated in a humid chamber. 24 flowers were employed per treatment and repletition, and the test was repeated in each case three times.

Pathogen: Erwinia amylovora 385 was grown on agar plates. The apple blossom was inoculated with a suspension of 10⁶ bacteria/ml.

Plant Treatment Composition:

1 part by weight of a solid formulation comprising

63.75% by weight of calcium formate

18.75% by weight of citric acid

16.73% by weight of calcium chloride

0.77% by weight of wetting agent and anti-dust agent were dissolved in 50 parts by weight (P 2%) of tap water and the solution was used for the following treatment. The pH was 4.1.

Treatments:

To carry out the treatments, the blossom was sprayed with the respective solution one hour after the inoculation, the chamber was sealed, and the contents were incubated for 6 days at 20 to 23° C.

(1-3) Water as the control

(4-6) 0.06% streptomycin (for comparison)

(7-9) P 2%

Evaluation

The infection level of the control which had been treated with tap water was 95.5%, 95.5% and 91.3% in tests 1 to 3, the efficacy of 0.06% streptomycin was 84.6%, 89.5% and 95.5% in tests 4 to 6. The efficacy of P 2% in tests 7 to 9 was in each case 100.0%.

What we claim is:

1. An aqueous plant treatment composition comprising at least 85% by weight of water, 0.1% by weight to 3% by weight of calcium salts, based on calcium oxide, and 0.15% by weight to 4.5% by weight of formates, based on formic acid, the pH based on standard conditions being 3.0 to 6.5, with the exception of those aqueous plant treatment compositions which are obtainable by suitably diluting formulations comprising 70 to 80% by weight of calcium formate, 14 to 18% by weight of calcium chloride and 1 to 15% by weight of citric acid, and with the the further exception of those plant treatment compositions which are obtainable by suitably diluting those concentrates which have a total calcium content of 260 g/l, based on calcium oxide, and which furthermore comprise 1.5% by weight of manganese in the form of manganese salts and 0.5% of zinc in the form of zinc salts and which have a pH of 5.5.

2. The aqueous plant treatment composition as claimed in claim 1, characterized in that the molar ratio of formates to calcium is between 1:4 and 4:1.

3. A formulation comprising between 5 and 40% by weight of calcium salts, based on calcium oxide, between 5 and 70% by weight of inorganic formates, based on formic acid, and from 0.5 to 70% by weight of an organic acid which is essentially solid under standard conditions or an acidic salt with a pKa of from 2 to 6, with the exception of formulations comprising from 70 to 80% by weight of calcium formate, 14 to 18% by weight of calcium chloride and 1 to 15% by weight of citric acid.

4. The formulation as claimed in claim 3, characterized in that it comprises from 10 to 98% by weight of calcium formate.

5. The formulation as claimed in either of claims 3 and 4, characterized in that it additionally comprises hygroscopic substances and/or humectants.

6. The formulation as claimed in any of claims 3 to 5, characterized in that it comprises between 50 and 80% by weight of calcium formate and over 15% by weight of a polybasic organic acid, and essentially liquid concentrates obtainable therefrom and aqueous plant treatment compositions obtainable therefrom.

7. A concentrate, obtainable by mixing in each case one part by weight of a formulation as claimed in any of claims 3 to 6 and 0.2 to 20 parts by weight of water, with the exception of concentrates which have a total calcium content of 260 g/l, based on calcium oxide, and which furthermore comprise 1.5% by weight of manganese in the form of manganese salts and 0.5% of zinc in the form of zinc salts and which have a pH of 5.5.

8. The use of the aqueous plant treatment compositions as claimed in claim 1, 2 or 7, of the formulations as claimed in any of claims 3 to 6 or of the concentrates as claimed in claim 7, and of the aqueous plant treatment compositions, formulations and concentrates excepted in each of claims 1 to 7 for preventing and/or controlling fire blight.

9. Method for controlling the bacterium Erwinia amylovora, characterized in that the bacterium is brought into contact with aqueous plant treatment compositions as claimed in claim 1, 2 or 7, formulations as claimed in any of claims 3 to 6 or concentrates as claimed in claim 7, or with the aqueous plant treatment compositions, formulations and concentrates excepted in each case in claims 1 to 7.

10. The use of calcium formate or organic acids or acidic salts for the preparation of aqueous plant treatment compositions as claimed in claim 1, 2 or 7, of formulations as claimed in any of claims 3 to 6 or of concentrates as claimed in claim 7.
